(19) United States
(12) Patent Application Publication
(10) Pub. No.: US 2006/0217558 A1
(43) Pub. Date: Sep. 28, 2006

(54) Method for producing 1, 3-dialkylpyridinium oligomers and related compounds using a solid support

(76) Inventor: Marcel Jaspars, Old Aberdeen (GB)

Correspondence Address: LICATA & TYRRELL P.C., 66 E. MAIN STREET, MARLTON, NJ, 08053, US

(21) Appl. No.: 10/561,496

(22) PCT Filed: Jun. 21, 2004
(86) PCT No.: PCT/GB04/02666

§ 371 Date: Jun. 9, 2006

(30) Foreign Application Priority Data
Jun. 19, 2003 (GB) 0314341.9

(51) Int. Cl. C07D 401/02; C40B 40/04

(52) U.S. Cl. 546/255

(57) ABSTRACT

A method of producing a linear di-substituted pyridinium compound of the formula NC₅R₄—R′-[-Q⁺NC₅R₄—R′—]ₙ—X using a solid support, wherein n is an integer, R is selected from hydrogen, hydroxyl, and substituted or unsubstituted alkyl, alkoxy, aryl, alkaryl, aralkyl, and alkenyl groups, R′ is a first linking group, and Q- and X are, respectively, a counter ion and a group which can react with the solid support.

The present invention relates to a method for producing a di-substituted pyridinium compound.

Dialkylpyridinium compounds, (Di-APS), in particular 1,3-APS oligomers, are known to be produced by sponges, for example the Haploscerid genera such as Haliclona, Amphimedon and Callyspongia, as part of their chemical defences, and have potentially useful biological properties. Diverse biological activities have been identified for different 1,3-APS compositions, including cytotoxicity, neurotoxicity and inhibition of action potentials, stimulation of transmitter release, inhibition of K⁺ conductances, and anticholinesterase activity. At least some of these observed actions of 1,3-APS compositions relate to the pore forming or membrane lesion effects of these compounds, which properties may be useful, for example, in the transfection of cells with genetic material.

Naturally occurring 1,3-APS compounds are produced as cocktails of different 1,3-APS compounds, from which individual 1,3-APS compounds are difficult to isolate, due to the same basic structure and very similar molecular weights of the different 1,3-APS compounds. However, it is desirable to be able to isolate different individual 1,3-APS compounds, in order to determine their different biological activities, for example with regard to the effect of degree of polymerisation, and length and rigidity of linking chains.

An alternative approach to isolating individual naturally occurring Di-APS compounds, is to synthesise these compounds by laboratory methods. Di-APS compounds generally occur as high molecular weight linear oligomers, having a molecular weight ranging from 1 KDa to greater than 25 KDa, having varying lengths of aliphatic chains linking the pyridine units. However, model linear Di-APS compounds closely analogous to the natural products remain to be synthesized by laboratory methods in a controllable fashion.

Previous studies have succeeded in producing linear 1,3-APS oligomers, but the methods employed meant that the product obtained consisted of a mixture of linear and cyclic oligomers with a wide range of molecular weights (see for example, Davies-Coleman, M. T.; Faulkner, D. J. J. Org. Chem. 1993, 58, 5925-5930). The approach used in these studies was to synthesize the 3-substituted pyridine monomer and introduce a good leaving group, for example bromide, at the end of the alkyl chain. This monomer was then refluxed to initiate polymerisation. Alternative methods based on this strategy incorporate an ether linkage in the linking chain (Gil, L. et al Tetrahedron Lett. 1995, 36, 2059-2062), or form cyclic dimers (Morimoto, Y.; Yokoe, C. Tetrahedron Lett. 1997, 38, 8981-8984) or tetramers with short linking chains (Shinoda, S. et al, Chem. Commun. 1998, 181).

A further study for producing macrocyclic 1,3-APS compounds is disclosed in Kaiser, A. et al., J. Am. Chem. Soc. 1998, 120, 8026-8034, which involves the formation and subsequent reaction of an N-(2,4-dinitrobenzene) pyridinium salt (Zinke salt) to yield linear 1,3-APS compounds. However, this method has certain disadvantages, in that it is relatively complex, and has only been shown to work for small macrocyclic oligomers.

Accordingly, there still exists a need for a reliable method for synthesizing linear di-substituted pyridinium compounds, for example di-APS oligomers and polymers. The present invention seeks to provide such a method, which overcomes the aforementioned disadvantages of previous methods.

According to the present invention there is provided a method of producing a linear di-substituted pyridinium compound, the method comprising the steps of:

(a) attaching a first 2, 3, or 4-substituted pyridine compound of the formula NC₅R₄—R′—X (1) to a solid support, to form a compound of the formula NC₅R₄—R′—Y-SUPPORT (2), wherein SUPPORT represents the solid support, R is selected from hydrogen, hydroxyl, and substituted or unsubstituted alkyl, alkoxy, aryl, alkaryl, aralkyl, and alkenyl groups, R′ is a first linking group, X is a group which can react with the solid support to attach the first pyridine compound to the support, and Y is absent or is a second linking group;

(b) forming a di-substituted pyridine compound of the formula ⁻A-⁺NC₅R₄—R′-Z (3) from a second 2, 3, or 4-substituted pyridine compound of formula (1), which second pyridine compound may be the same as or different to the first pyridine compound, wherein A is a protecting group, and Z is a leaving group;

(c) reacting the compound of formula (2) formed in step (a) with the compound of formula (3) formed in step (b), to form a di-substituted pyridinium compound of the formula ⁻A-⁺NC₅R₄—R′-[⁻Q⁺NC₅R₄—R′—]ₙY-SUPPORT (4), wherein Q⁻ is a counter ion and n=1;

(d) optionally, repeating step (c) as many times as required to obtain a compound of formula (4) wherein n is an integer of 2 or greater; and

(e) detaching the compound of formula (4) from the solid support, and removing the protecting group A to form a di-substituted pyridinium compound of the formula NC₅R₄—R′-[⁻Q⁺NC₅R₄—R′—]ₙ—X (5), wherein n is an integer, and Q⁻ and X are a counter ion and a group which can react with the solid support to attach the first pyridine compound to the support respectively, which may the same or different as Q and X defined above in steps (c) and (a) respectively.

Thus, during steps (a) to (d) of the method of the present invention, one end of the compound which is to become the product compound of formula (5) is bound to a solid support, thereby removing it from the reaction equilibrium, and substantially eliminating cyclisation. The use of protecting and leaving groups per the method of the present invention allows for the controlled addition of monomer units without macrocycle formation occurring in solution. The method of the present invention can be used to produce di-substituted pyridinium compounds in high rates of conversion, and in high yields. The method of the present invention also has the advantage that a variety of linking groups may be used, which may incorporate different functionalities, for example functionalities which can restrict the conformational space accessible by the di-substituted pyridinium compound, such as a double bond or cyclopropyl ring, or groups which allow the attachment of fluorescent labels, so that the di-substituted pyridinium compound can be localised intracellularly and in cell membranes.

Herein the term “di-substituted pyridinium compound” can refer to either a 1,2-, 1,3-, or 1,4-substituted pyridinium compound. However, the preferred products of the method of the present invention are 1,3-substituted pyridinium compounds, in particular linked 1,3-dialkyl pyridinium compounds.

Although the method of the present invention is suitable for producing oligomers and polymers potentially having a wide range of degrees of polymerisation, and hence molecular weights, for example n=1 to 20 in formula (5) above, it is particularly useful for producing oligomers having higher degrees of polymerisation, for example n=20 to 100 in formula (5) above.

Referring to the steps of the method of the present invention in turn, in step (a) a first 2,3, or 4-substituted, preferably 3-substituted, pyridine compound of the formula NC₅R₄—R′—X (1) is attached to a solid support, to form a compound of the formula NC₅R₄—R′—Y-SUPPORT (2), wherein SUPPORT represents the solid support, R is selected from hydrogen, and substituted or unsubstituted alkyl, aryl, alkaryl, aralkyl, and alkenyl groups, R′ is a first linking group, X is a group which can react with the solid support to attach the first pyridine compound to the support, and Y is absent or is a second linking group. Each R group is preferably a hydrogen atom.

Compounds of formula (1) are commercially available, or may be synthesised as is known in the art. Thus, for example, a compound of formula (1) may be synthesised by reaction of a compound of formula Z′-R″—X with a suitable pyridine compound, with protection of the X— group as necessary, wherein R″ is a linking group and Z′ is a suitable leaving group. For example, a compound of formula (1) may be prepared by reacting Br—R″—OH (i.e. Z′=Br and X=OH) with t-butyldimethyl-chlorosilane (TBDMSCl) to form Br—R″-OTBDMS, which may be reacted with 2,3, or 4-methylpyridine (2,3, or 4-picoline) with deprotection of the X group to form NC₅H₄—R—OH, wherein R′ is as defined above, i.e. a linking group equivalent to R″ plus one carbon atom. Of course, protecting groups other than TBDMSCl may be used, as will be apparent to those skilled in the art.

As referred to above, the method of the present invention is advantageous in that a variety of linking groups may be used, which may incorporate different functionalities. Preferred R′ groups have no terminal carbon atoms, which further restricts the possibility of cyclisation occurring.

Thus, in formulas (1) to (5) above, each group R′ may be the same or different and selected from an alkylene group (for example, a group —(CH₂)ₘ—, wherein m is an integer from 2 to 12, preferably from 6 to 10), an alkenyl-containing group (for example, a group having from 2 to 12 carbon atoms, preferably from 6 to 10 carbon atoms, containing one or more alkenyl groups, e.g. cis- or trans-2-butenyl, 3-hexenyl, 2,5-hepten-di-yl, and 4-octenyl groups), a cyclopropanyl-containing group (for example, cis- or trans- —(CH₂)ₚ-cyclopropanyl-(CH₂)_{q}— wherein p and q are the same or different and are integers from 1 to 4, preferably 1 or 2). As discussed above, R′ may also be a group which comprises a fluorescent label, so that the di-substituted pyridinium compound can be localised intracellularly and in cell membranes (for example, a linking group R′ having a pendant alcohol group, for attachment of a fluorescent group).

Where appropriate, cis- or trans-isomerism may be introduced into linking group R′ as is known in the art (for example, Morimoto, Y.; Yokoe, C. Tetrahedron Lett. 1997, 38, 8981-8984). A double bond may be converted into a cyclopropyl ring using carbene chemistry.

In the compound of formula (1), X is a group which can react with the solid support to attach the compound of formula (1) to the support. Thus, the particular group X to be used in a particular compound of formula (1) will depend upon the particular solid support being used. Thus, suitable X groups include hydroxyl, carboxyl, thiol, and amine groups. However, X is preferably a hydroxyl group, and particularly preferred compounds of formula (1) are pyridines 3-substituted by a hydroxyalkyl group, i.e. compounds of the formula NC₅R₄— (CH₂) ₙ—OH, wherein n is as defined above.

The solid support used in the present invention may be any suitable support to which the compound of formula (1) may be attached. Preferred solid support materials are organic resins having functionality which can react with group X of the compound of formula (1), described above. Particularly preferred solid support materials are trityl chloride and functionalised polystyrene resins (for example, Merryfield resins, i.e. chloromethylstyrene-divinylbenzene resins).

Group Y in formula (2) is absent or a second linking group, depending on the particular reaction which occurs between the compound of formula (1) and the solid support to form the compound of formula (2). Thus, group X in formula (1) may be eliminated, or, for example, in the case of a compound of formula (1) in which X is a hydroxyl group, the compound of formula (2) may have the more specific formula NC₅R₄—R′—O-SUPPORT, i.e. Y may be an oxygen atom.

In step (b) of the method of the present invention, a disubstituted pyridine compound of the formula ⁻A-⁺NC₅R₄—R′-Z (3) is formed from a second 2, 3 or 4-substituted pyridine compound of formula (1), which second pyridine compound may be the same as or different to the first pyridine compound, wherein A is a protecting group, and Z is a leaving group. Step (b) is independent of step (a), and accordingly may be performed before, after, or simultaneously with step (a).

Step (b) itself thus includes two steps: a first step of converting group X to a leaving group Z, for reaction with the nitrogen atom of the pyridine group of the compound of formula (2), and a second step of protecting the nitrogen atom of the second pyridine compound, with a protecting group A.

In step (b), group X may be converted to a suitable leaving group Z as is known in the art. For example, as a hydroxyl group, X may be converted to a mesyl(methanesulphonyl) group by reaction with mesyl chloride. Other suitable leaving groups will be apparent to those skilled in the art.

The nitrogen atom of the second pyridine compound of formula (1) used in step (b) may be protected by convertion to the N-oxide as is known in the art, i.e. protecting group A is oxygen, for example by reaction of the nitrogen atom of the pyridine group with a peracid, for example m-chloroperbenzoic acid. Alternatively, the nitrogen atom may be protected by formation of the borane, i.e. A is BH₃—.

In step (c) of the method of the present invention, the compound of formula (2) formed in step (a) is reacted with the compound of formula (3) formed in step (b), to form a di-substituted pyridinium compound of the formula ⁻A-⁺NC₅R₄—R′-[⁻Q⁺NC₅R₄—R′—]ₙY-SUPPORT (4), wherein Q⁻ is a counter ion and n=1. Thus, in step (c) two pyridine-containing monomer units are combined to form a dimeric di-substituted pyridinium compound, the leaving group Z of the compound of formula (3) reacting with the nitrogen atom of the pyridine group of the compound of formula (2). The reaction may take place on heating, in the presence of a suitable counter ion Q⁻, for example a chloride, iodide, or other suitable counter ion.

In step (d) of the method of the present invention, step (c) is optionally repeated as many times as required to obtain a compound of formula (4) wherein n is an integer of 2 or greater. If the desired di-substituted pyridinium compound end product is a dimer (i.e. a compound of formula (5) in which n=1), then no repetitions of step (c) are required. However, as discussed above, the method of the present invention is particularly useful for producing oligomers having higher degrees of polymerisation, for example n=20 to 100 in formula (5) above, which will of course require the repetition of step (c) the appropriate number of times.

In preferred embodiments of the present invention, chain extension to produce oligomers having a higher degree of polymerisation may be accelerated by releasing oligomers having a lower degree of polymerisation from the solid support as compounds having the formula (5), and reintroducing them as reagents as an alternative to, or in addition to, the second pyridine compound used in step (b). Thus, in these preferred embodiments, a compound of formula NC₅R₄—R′-[⁻Q⁺NC₅R₄—R′—]ₙ—X (5) may be converted to a compound of formula A⁻-⁺NC₅R₄—R′-[⁻Q⁺NC₅R₄—R′-]ₙ-Z (5a) per step (b), and the compound of formula (5a) may then be reacted with the compound of formula (2) formed in step (a) or (c), per step (d).

In step (e) of the method of the present invention, the compound of formula (4) is detached from the solid support, and reduced to form a di-substituted pyridinium compound of the formula NC₅R₄—R′-[⁻Q⁺NC₅R₄—R′—]ₙ—X (5), wherein n is an integer, and Q⁻ and X are a counter ion and a group which can react with the solid support to attach the first pyridine compound to the support respectively. Q⁻ and X of step (e) may be the same or different as Q⁻ and X defined above with reference steps (a) and (c) respectively. The compound of formula (4) may be detached from the solid support according to the particular solid support being used. Thus, in the case of a functionalised trityl chloride resin solid support, the compound of formula (4) may be detached using a strong acid. For example, if the strong acid used to detach the formula (4) from the solid support is hydrochloric acid, then counter ion Q⁻ will be chloride. The protecting group A can be removed as is known in the art, for example by reducing the N-oxide to a nitrogen atom where A in formula (4) is oxygen.

An embodiment of the present invention will now be described in detail.

Reaction Scheme 1 below describes a preferred method for producing a preferred linear 1,3-alkylpyridinium compound of the present invention. 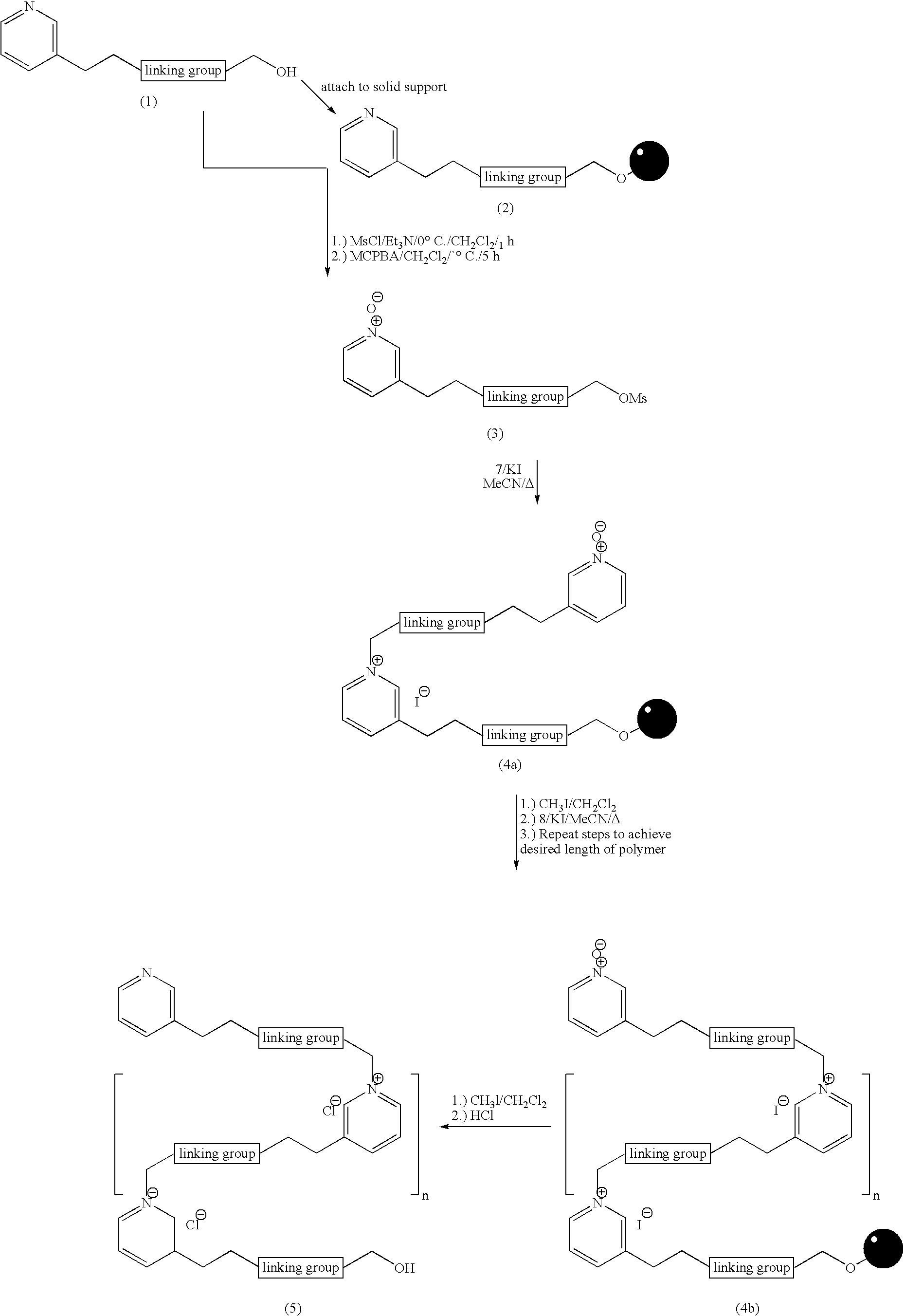
wherein:

Ms=methanesulphonyl(mesyl)

Et=ethyl

MCPBA=m-chloroperbenzoic acid

Referring to Scheme 1,3-substituted pyridine compound (1) is formed by firstly reacting Br—R″—OH with t-butyldimethyl-chlorosilane (TBDMSCl) to form Br—R″-OTBDMS, wherein R″ is a suitable linking group (described hereinabove). The Br—R″-OTBDMS is then reacted with 3-methylpyridine (3-picoline) with deprotection of the X group to form NC₅R₄—R′—OH (3-substituted pyridine compound (1)), wherein R′ is a linking group equivalent to R″ plus one carbon atom.

A first molecule of 3-substituted pyridine compound (1) is then attached to a trityl chloride resin solid support, to form compound (2).

Before, after, or simultaneously with the formation of compound (2), a second molecule of 3-substituted pyridine compound (1) is reacted firstly with mesyl chloride, and secondly with m-chloroperbenzoic acid to form compound (3) of formula ⁻O—⁺NC₅R₄—R′—O-Ms, wherein Ms is a mesyl group.

Next, compound (2) is reacted with one equivalent of compound (3) to form 1,3-alkylpyridinium compound (4a) having the formula ⁻O—⁺NC₅R₄—R′—⁻I⁺NC₅R₄—R′—O-SUPPORT, i.e. two pyridine-containing monomer units are combined to form a dimeric 1,3-alkylpyridinium compound, the -Ms leaving group of compound (3) reacting with the nitrogen atom of the pyridine group of compound (2). This step is repeated the appropriate number of times to obtain an 1,3-alkylpyridinium compound end product having the desired degree of polymerisation, i.e. compound (2) is reacted with 1,3-alkylpyridinium compound (4a) to produce compound (4b) of formula ⁻O—⁺NC₅R₄—R′—[⁻I⁺NC₅R₄—R′—]ₙO-SUPPORT, wherein n is preferably 20 to 100.

As discussed above, in preferred embodiments of the present invention, chain extension to produce oligomers having a higher degree of polymerisation may be accelerated by releasing oligomers having a lower degree of polymerisation from the solid support as compounds having the formula (5), and reintroducing them as reagents as an alternative to, or in addition to, the second pyridine compound used in step (b). Thus, in these preferred embodiments, a compound of formula NC₅R₄—R′-[⁻Q⁺NC₅R₄—R′—]ₙ—X (5) may be converted to a compound of formula O⁻—⁺NC₅R₄—R′-[⁻Q⁺NC₅R₄—R′-]ₙ-Z (5a) per step (b), and the compound of formula (5a) may then be reacted with the compound of formula (2) formed in step (a) or (c), per step (d).

Finally in Scheme 1, compound (4b) is detached from the solid support, and reduced to form 1,3-alkylpyridinium compound (5) of formula NC₅R₄—R′—[⁻Cl⁺NC₅R₄—R′—]ₙ—OH using hydrochloric acid, as shown. The counter ion in final product (5) is thus chloride.

As referred to above, the method of the present invention is advantageous in that a variety of linker groups may be used, which may incorporate different functionalities.

Reaction Scheme 2 below describes a method for producing a compound from which starting material NC₅R₄—R′—X used in step (a) of the present invention may in turn be produced, in which R′ is an alkenyl-containing linking group having either cis- or trans-isomerism. 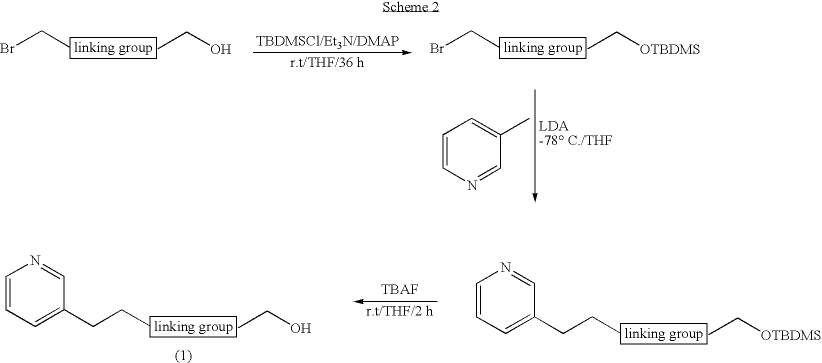
wherein:

TBDMS=t-butyldimethylsilyl

DMAP=-4-dimethylaminopyridine

THF=tetrahydrofuran

LDA=lithium diisopropylamine

TBAF=t-butylammonium fluoride

Reaction Scheme 3 below describes a method for producing a compound from which starting material NC₅R₄—R′—X of formula (1) used in step (a) of the present invention may in turn be produced, in which R′ is an alkylene linking group having a pendant alcohol group to which a fluorescent group may be attached. 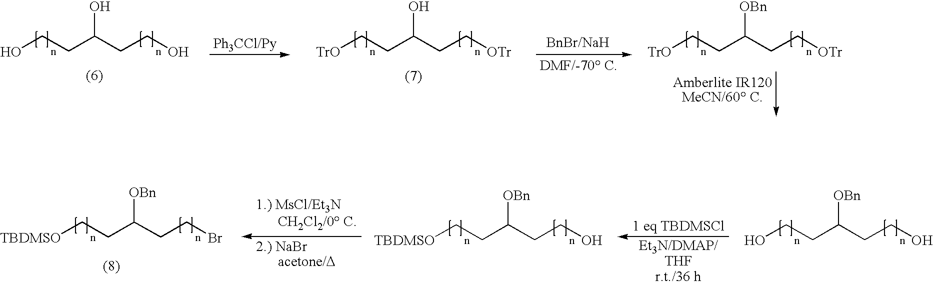
wherein:

Ph=phenyl

Py=pyridine

Tr=trityl

Bn=benzyl

As shown in Scheme 3, triol (6) is differentially protected to form alcohol (7), which is subsequently converted to bromide (8), which can then in turn be used to form starting material NC₅R₄—R′—X of formula (1) used in step (a) of the present invention.

Fluorescent labels, available commercially, can be reacted with the monomer or oligomer, depending on the reaction conditions necessary. To avoid cell damage, the excitation wavelength of the fluorescent label is preferably above 350 nm, and the emission wavelength should preferably be greater than 80 nm higher, to permit ratiometric measurements. Suitable fluorescent labels include fluorescein, lissamine, and rhodamine based compounds, although other fluorescent materials may be used according to conditions. Such a linking group having a fluorescent group would preferably be incorporated into the 1,3-APS compound end product only once or twice.

1-27. (canceled)

28. The method of producing a linear di-substituted pyridinium compound, the method comprising the steps of:
(a) attaching a first pyridine compound selected from the group consisting of a 2-substituted pyridine compound, a 3-substituted pyridine compound and 4-substituted pyridine compound of the formula NC₅R₄—R′—X (1) to a solid support, to form a compound of the formula NC₅R₄—R′—Y-SUPPORT (2), wherein SUPPORT represents the solid support, R is selected from the group consisting of hydrogen, hydroxyl, and substituted and unsubstituted alkyl, alkoxy, aryl, alkaryl, aralkyl, and alkenyl groups, R′ is a first linking group, X is a group which can react with the solid support to attach the first pyridine compound to the support, and Y is selected from the group consisting of a direct bond and a second linking group;
(b) forming a di-substituted pyridine compound of the formula ⁻A-⁺NC₅R₄—R′-Z (3) from a second pyridine compound selected from the group consisting of a 2-substituted pyridine compound of formula (1), a 3-substituted pyridine compound of formula (1) and a 4-substituted pyridine compound of formula (1), which second pyridine compound may be selected from the group consisting of the same pyridine compound as the first pyridine compound and a different pyridine compound from the first pyridine compound, wherein A is a protecting group, and Z is a leaving group;
(c) reacting the compound of formula (2) formed in step (a) with the compound of formula (3) formed in step (b), to form a di-substituted pyridinium compound of the formula ⁻A-⁺NC₅R₄—R′-[⁻Q⁺NC₅R₄—R′—]ₙY-SUPPORT (4), wherein Q⁻ is a counter ion and n=1;
(d) optionally, repeating step (c) as many times as required to obtain a compound of formula (4) wherein n is an integer of ≧2; and
(e) detaching the compound of formula (4) from the solid support, and reducing to form a di-substituted pyridinium compound of the formula NC₅R₄—R′-[⁻Q⁺NC₅R₄—R′—]ₙ—X (5), wherein n is an integer, and Q⁻ and X are a counter ion and a group which can react with the solid support to attach the first pyridine compound to the support respectively, which may be selected from the group consisting of the same Q and X as defined above in steps (c) and (a) respectively and different Q and X as defined above in steps (c) and (a) respectively.

29. The method according to claim 28 wherein each R group is hydrogen.

30. The method according to claim 28 wherein in step (d), step (c) is repeated such that in formula (5) n=20 to 100.

31. The method according to claim 28 wherein the compound of formula (1) is prepared by reaction of a compound of formula Z′-R″—X with a pyridine compound, with protection of the X— group as necessary, wherein R″ is a linker group and Z′ is a suitable leaving group.

32. The method according to claim 31 wherein the compound of formula (1) is prepared by reacting Br—R″—OH with t-butyldimethyl-chlorosilane (TBDMSCl) to form Br—R″-OTBDMS, which is reacted with a compound selected from the group consisting of 2-methylpyridine (2-picoline), 3-methylpyridine (3-picoline) and 4-methylpyridine (4-picoline) with deprotection of the X group to form NC₅R₄—R′—OH.

33. The method according to claim 28 wherein linker group R′ has no terminal carbon atoms.

34. The method according to claim 28 wherein each group R′ is the same and is selected from an alkylene group, an alkenyl-containing group, and a cyclopropanyl-containing group.

35. The method according to claim 34 wherein each group R′ is selected from the group consisting of a group —(CH₂)ₘ—, wherein m is an integer from 2 to 12, a group having from 2 to 12 carbon atoms containing at least one alkenyl group, a cis- —(CH₂)ₚ-cyclopropanyl-(CH₂)_{q}— group wherein p and q are the same or different and are integers from 1 to 4, and a trans- —(CH₂)ₚ-cyclopropanyl-(CH₂)_{q}— group wherein p and q are the same or different and are integers from 1 to 4.

36. The method according to claim 28 wherein each group R′ is different and is selected from an alkylene group, an alkenyl-containing group, and a cyclopropanyl-containing group.

37. The method according to claim 36 wherein each group R′ is selected from a group —(CH₂)ₘ—, wherein m is an integer from 2 to 12, a group having from 2 to 12 carbon atoms containing at least one alkenyl group, a cis- —(CH₂)ₚ-cyclopropanyl-(CH₂)_{q}— group wherein p and q are the same or different and are integers from 1 to 4, and a trans- —(CH₂)ₚ-cyclopropanyl-(CH₂)_{q}— group wherein p and q are the same or different and are integers from 1 to 4.

38. The method according to claim 28 wherein R′ is selected from the group consisting of a group which comprises a fluorescent group and a group to which a fluorescent group can be attached.

39. The method according to claim 38 wherein R′ has a pendant alcohol group for attachment of a fluorescent group.

40. The method according to claim 28 wherein in formula (1) X is selected from the group consisting of hydroxyl, carboxyl, thiol, and amine groups.

41. The method according to claim 40 wherein X is a hydroxyl group.

42. The method according to claim 41 wherein the compound of formula (1) is a compound of the formula NC₅R₄—(CH₂)ₙ—OH.

43. The method according to claim 28 wherein the solid support material comprises an organic resin having functionality which can react with group X of the compound of formula (1).

44. The method according to claim 43 wherein the solid support material comprises a member selected from the group consisting of trityl chloride and a functionalised polystyrene resin.

45. The method according to claim 28 wherein group Y in formula (2) is an oxygen atom.

46. The method according to claim 28 wherein in step (b) group X is converted to a mesyl(methanesulphonyl) group by reaction with mesyl chloride.

47. The method according to claim 28 wherein A is selected from the group consisting of oxygen and BH₃—.

48. The method according to claim 47 wherein A is oxygen, and the nitrogen atom of the second pyridine compound of formula (1) used in step (b) is converted to the N-oxide by reaction of the nitrogen atom of the pyridine group with a peracid.

49. The method according to claim 48 wherein the peracid comprises m-chloroperbenzoic acid.

50. The method according to claim 28 wherein counter ion Q⁻ in step (c) is an iodide ion.

51. The method according to claim 28 wherein oligomers having the formula (5) are released from the solid support and reintroduced as reagents as an alternative to the second pyridine compound used in step (b).

52. The method according to claim 51 wherein a compound of formula NC₅R₄—R′-[⁻Q⁺NC₅R₄—R′—]ₙ—X (5) is converted to a compound of formula ⁻A-⁺NC₅R₄—R′-[⁻Q⁺NC₅R₄—R′-]ₙ-Z (5a) per step (b), and the compound of formula (5a) is then reacted with the compound of formula (2) formed in step (a) or (c), per step (d).

53. The method according to claim 28 wherein oligomers having the formula (5) are released from the solid support and reintroduced as reagents in addition to the second pyridine compound used in step (b).

54. The method according to claim 53 wherein a compound of formula NC₅R₄—R′-[⁻Q⁺NC₅R₄—R′—]ₙ—X (5) is converted to a compound of formula ⁻A-⁺NC₅R₄—R′-[⁻Q⁺NC₅R₄—R′—]ₙ-Z (5a) per step (b), and the compound of formula (5a) is then reacted with the compound of formula (2) formed in step (a) or (c), per step (d).

55. The method according to claim 28 wherein the compound of formula (4) is detached from the solid support, and reduced to form a di-substituted pyridinium compound of the formula NC₅R₄—R′-[⁻Q⁺NC₅R₄—R′—]ₙ—X (5) using an acid.

56. The method according to claim 55 wherein the acid is hydrochloric acid, and counter ion Q⁻ is chloride.

57. The method according to claim 28 wherein the di-substituted pyridinium compound is a linked dialkyl pyridinium compound.
